(19) United States
(12) Patent Application Publication
(10) Pub. No.: US 2005/0164316 A1
(43) Pub. Date: Jul. 28, 2005

(54) Biophotonic sensors and methods of use thereof

(76) Inventor: Terry L. Bray, Birmingham, AL (US)

Correspondence Address: BARNES & THORNBURG, 11 SOUTH MERIDIAN, INDIANAPOLIS, IN, 46204, US

(21) Appl. No.: 10/480,963

(22) PCT Filed: Jun. 26, 2002
(86) PCT No.: PCT/US02/20287

Related U.S. Application Data
(60) Provisional application No. 60/301,380, filed on Jun. 27, 2001.

(51) Int. Cl. G01N 33/53; G01N 33/537; G01N 33/543; C12Q 1/66

(52) U.S. Cl. 435/7.92; 435/8

(57) ABSTRACT

The present invention provides novel biophotonic sensors that have molecular recognition with high sensitivity for target molecules. In one embodiment, the biophotonic sensors have capture moieties with high specificity for molecules of interest (target molecules) and biophotonic conjugates. The biophotonic conjugates exhibit a characteristic photonic activity only when a target molecule is bound. This characteristic photonic activity may include, but is not limited to, either a qualitative response or a measurable change in photonic characteristics upon interaction of the sensors with the target molecules. Methods are also provided for use of the biophotonic sensors to detect molecules of interest either in vitro, in vivo, or in situ.

### FIELD OF THE INVENTION

The present invention relates generally to biophotonic sensors and more particularly, to biophotonic sensors having both a binding site for target molecules and a biophotonic conjugate.

### BACKGROUND OF THE INVENTION

Sensing systems to probe physiological processes are widely used in research and medical laboratories worldwide. Biosensors in particular have proven capable of high specificity for target molecules. The most widely used biosensors are enzyme based, with commercial applications directed at blood-glucose measurement, pregnancy and fertility testing. However, translating a low level molecular recognition event into a measurable signal can be challenging. Biosensors based on molecular interaction are often limited by insensitivity except at relatively high concentrations, though an ion-channel switch-based biosensor has been used to detect picomolar concentrations of proteins. Recent advances have coupled photonics with biosensors to create a new class of biophotonic sensors that show promise in the non-invasive detection of blood-glucose levels, diabetes, cancer, and bilirubin levels in infants. Rutter, G A, et al., Proc. Nat'l. Acad. Sci. USA, 93: 5489-5494 (1996). These advances all demonstrate the potential that joining the specificity of molecular of recognition with the sensitivity of photonics can have for detection of biologically important molecules.

Biophotonics describes a general class of detection methods that couple biological-based recognition with photonic phenomena. Many different implementations of biophotonics have been utilized to study gene expression (Turner, A. P. F., Nature 387: 555-557 (1997)), adenosine triphosphate (ATP) production (Cornell, B. A. et al., Nature 387: 580-583 (1997)), and measurement of calcium levels in cells with high specificity and sensitivity. However, use of these methods for non- or minimally invasive sensing of molecular targets in vivo has been impractical. In addition to detection of physiological fluid components, a number of biophotonic systems have been developed to probe cellular processes. These include fluorescent and bioluminescent agents for use in reporter gene assays and determination of ATP. For example, U.S. Pat. Nos. 5,532,129 and 6,162,602 disclose modified synthetic nucleic acid polymers into which functional photonic properties are directly incorporated. These modified nucleic acid polymers are used for detecting hybridization to a specific DNA sequence.

Thus it would be desirable to provide a biophotonic sensor combining molecular recognition and biophotonic signaling for both in vivo and in vitro use. Such a device would provide detection of low levels of biomolecule and other molecular targets. It would also be desirable to provide methods for using such devices.

### SUMMARY OF THE INVENTION

The present invention provides novel biophotonic sensors that have molecular recognition with high sensitivity for target molecules. In one embodiment, the biophotonic sensors have a capture moiety with a high specificity for molecules of interest (target molecules) and a biophotonic conjugate. The capture moiety further has at least one component for binding the target molecule. Preferably, the capture moiety has at least two binding components. Furthermore, the biophotonic conjugate has at least two photonic components. The biophotonic conjugate exhibits a characteristic photonic activity only when a target molecule is bound. This characteristic photonic activity may include, but is not limited to, either a qualitative response or a measurable change in photonic characteristics upon interaction of the sensors with the target molecules.

In another embodiment, the photonic sensors of the present invention may be a single unit or multiple units. In an illustrative embodiment, the components of both the capture moiety and biophotonic conjugate are contained on a single biophotonic sensor unit. In a further embodiment, the biophotonic sensors can either be free in solution or bound to a solid matrix. The biophotonic sensors can be bound to the solid matrix by, but not limited to, hydrophobic interactions, hydrophilic interactions, ionic interactions or covalently bound. Non-limiting examples of solid matrices are multi-well plates, tubes and beads.

Methods for using the biophotonic sensors of the present invention are also provided. The biophotonic sensor is added to a sample, organism or mammal to determine if the desired target molecule is present. Additionally, if desired, the amount of the target molecule that is present in the sample may be determined. In one embodiment, the biophotonic sensor is initially in an inactive state, with minimal or no contact between the components of the biophotonic conjugate. Binding of the target molecule to the capture moiety produces the biophotonic sensor in an active state. In the active state the conformation of the sensor changes such that the components of the biophotonic conjugate will come into close proximity and/or contact with each other, producing photonic activity resulting in a photonic signal. The photonic signal is then collected by a device such as, but not limited to, fiber optic cables, arrays, waveguides or secondary reporting devices. In an alternate embodiment, the biophotonic sensor is initially in the inactive state in which the biophotonic conjugates form a complex prior to binding of the target molecule that deactivates, quenches or gives only background levels of the photonic activity of the biophotonic conjugate. Binding of the target molecule causes the inactive complex to dissociate, generating a biophotonic sensor in an active state.

Additional objects, advantages, and features of the present invention will become apparent from the following description and appended claims, taken in conjunction with the accompanying drawings.

### BRIEF DESCRIPTION OF THE DRAWINGS

The various advantages of the present invention will become apparent to one skilled in the art by reading the following specification and subjoined claims and by referencing the following drawings in which:

FIG. 1A is a schematic showing a biophotonic sensor on more than one unit;

FIG. 1B is a schematic showing a biophotonic sensor on a single unit;

FIG. 2 is a schematic showing the conversion of a biophotonic sensor on more than one unit bound to a solid matrix from an inactive state to an active state upon binding of a target molecule;

FIG. 3 is a schematic showing the conversion of a biophotonic sensor on a single unit bound to a solid matrix from an inactive state to an active state upon binding of a target molecule; and

FIG. 4 is a schematic showing the conversion of a biophotonic sensor from an inactive state to an active state upon bind of a target molecule.

### DETAILED DESCRIPTION OF THE PREFERRED EMBODIMENTS

The present invention provides novel biophotonic sensors that have molecular recognition with high sensitivity for target molecules. The biophotonic sensors of the present invention comprise at least one capture moiety which has a high specificity for molecules of interest (target molecules) and at least one biophotonic conjugate. The biophotonic conjugate exhibits a characteristic photonic activity only when a target molecule is bound. This characteristic photonic activity may include, but is not limited to, either a qualitative response or a measurable change in photonic characteristics upon interaction of the sensors with the target molecules.

In one embodiment, the capture moiety has a high affinity and specificity for a target molecule. The target molecule is any molecule of interest that can be bound by the binding components of the capture moiety. The target molecule can include individual molecules, molecular complexes or classes of molecules. Furthermore, the target molecule may be free in solution or it may be immobilized such as, but not limited to, a receptor, glycoprotein or oligosaccharide on a virus capsule or a cell membrane. Non-limiting examples of target molecules are nucleic acid molecules, proteins, enzymes, receptors, metals, coenzyme, small organic or inorganic molecules or any other compound of interest. The capture moiety is comprised of at least one binding component that interacts and/or binds the target molecule. Examples of binding components of the capture moiety include, but are not limited to, antibodies, antibody fragments, enzyme inhibitors, receptor-binding molecules, ligands, various toxins and the like.

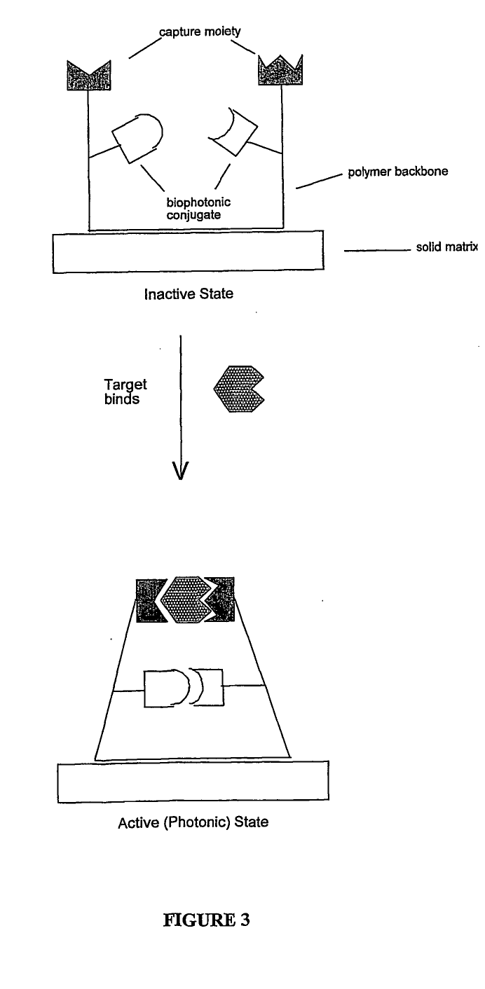

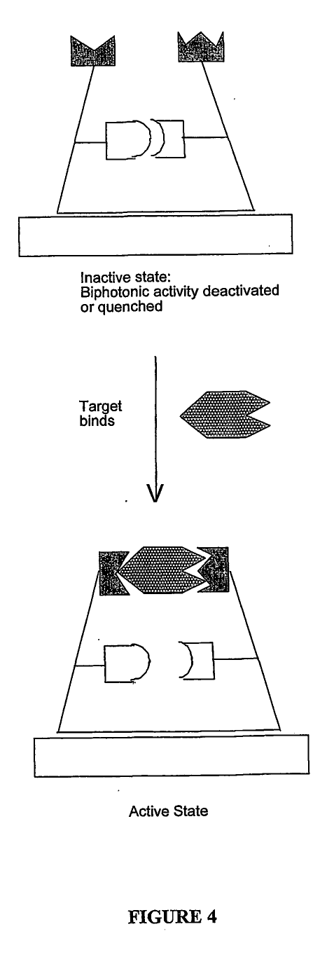

In another embodiment, the biophotonic sensors of the present invention comprise at least one biophotonic conjugate. The biophotonic conjugate preferably has at least two components which, when they interact with one another, produce a unique and measurable photonic activity. In a preferred embodiment, the biophotonic conjugate is a fluorescent, bioluminescent, or a chemiluminescent conjugate, and more preferably, a bioluminescent conjugate. In one aspect of the embodiment the system is active and the conjugate produces a signal, preferably fluorescent or luminescent (FIG. 3), when the components of the biophotonic conjugate are in close proximity or in contact with each other. Initially, in the absence of a target molecule, the components are separated so that either there is no signaling or minimal (i.e. background) signal. Upon binding of a target molecule, the components are brought together and a signal is generated. In an alternate aspect of the embodiment, when the components of the biophotonic conjugate are in close proximity or contact with each other, the system is inactive and no signal or only minimal signal is observed. Upon binding of the target molecule, the components are moved away from each other and a signal is observed (FIG. 4).

In an illustrative embodiment the biophotonic conjugate of the present invention is a fluorescent conjugate. The fluorescent biophotonic conjugates will have components that form appropriate donor and acceptor pairs which are capable of energy transfer by dipole coupling.

A chromophore refers to those groups which have favorable absorption characteristics, i.e., are capable of excitation upon irradiation by any of a variety of photonic sources. Chromophores can be fluorescing or non-fluorescing. Non-fluorescing chromophores typically do not emit energy in the form of photonic energy (hv₂). Thus they can be characterized as having a low quantum yield, which is the ratio of emitted photonic energy to adsorbed photonic energy, typically less than 0.01. A fluorescing chromophore is referred to as a fluorophore, and typically emits photonic energy at medium to high quantum yields of 0.01 to 1.

An acceptor chromophore for the purposes of the present invention is a fluorophore that is capable of accepting energy transfer from a donor chromophore and producing an emission spectrum. Because energy transfer by dipole coupling can typically occur when there is an overlap in the emission spectrum of the donor and the excitation spectrum of the acceptor, a “suitable” acceptor typically has an excitation spectrum in the longer wavelengths than its corresponding suitable donor. In this regard, donors and acceptors can be paired for capacity to transfer energy on the basis of overlapping donor emission and acceptor excitation spectra. Therefore, potentially any chromophore can be paired with another chromophore to form an acceptor-donor pair, so long as the two chromophores have different emission spectrums, and have sufficiently overlapping donor emission and acceptor excitation spectra to affect energy transfer.

A non-fluorescent donor producing fluorescent re-emission in the acceptor group is an extremely valuable property. The non-fluorescing donor in a composition of the present invention provides the particular advantage of a low or absent level of emission by the donor, thereby not contributing to background or the detectable emitted light in a donor-acceptor system. Thus, non-fluorescent donors allow for very low background and are particularly preferred. A multiple donor system comprised of such non-fluorescent chromophores would have very little inherent fluorescent background. This property overcomes a major limitation that has severely limited practical uses of fluorescent energy transfer in biophotonic sensor applications. It also opens opportunity to create more useful photonic mechanisms and applications. With regard to unique properties in acceptors, most preferred are acceptors with the highest quantum yields, or with other properties that increase the signal-to-noise ratio between specific acceptor emissions and the background (nonspecific) emissions attributable to the donor. Examples of approaches to reduce the signal-to-noise ratio include using donors having lower emission, preferably non-fluorescing donors, selection of acceptor-donor pairs in which the spectral distance between the emission spectrum of the donor and acceptor is maximized, and preferably selected as to be non-overlapping.

Multiple donors groups (fluorescent and non-fluorescent) transferring energy to a single or smaller number of acceptor groups can be used. Generally, multiple donors transfer to a single acceptor group, but under some conditions and for certain photonic mechanisms more than one acceptor group may be used. The preferred arrangements are those involving the non-fluorescent donors, which provide the important advantage of a low background extended energy transfer process. Other preferred arrangements involve multiple fluorescent donors, excited in the visible region, which transfer to an acceptor(s) which re-emits in the infrared region. This is a useful mechanism because the infrared emission can be detected by optoelectronic devices which are much less sensitive to background fluorescence produced in the visible region.

Chromophore groups with strong quenching properties can also be used to prevent fluorescent emission by the acceptor group. Preferably quenching chromophores are used in biophotonic sensors in which the inactive configuration has the components of the biophotonic conjugate in contact or close proximity with each other. In this embodiment, the present invention contemplates the use of a quencher chromophore (or quencher), that has the capacity to accept, like an acceptor, the transfer of energy by dipole coupling, but does not have significant emission. Although similar in properties to a non-fluorescing donor, the term quencher refers to a non-fluorescing chromophore that is configured to draw the energy potential away from an excited acceptor so that the acceptor does not emit, i.e., the acceptor is quenched.

The mechanism for energy transfer to a quenching chromophore is the same as for donor-donor or donor-acceptor transfer, namely dipole coupling, and therefore is subject to the same requirements as described herein relating to transfer distances and optimum pairing configurations. It is important to point out that the various arrangements and configurations of donor, acceptor, and quencher groups described above can be achieved by either incorporating them within the biophotonic sensor consisting of either a single unit or multiple units.

In an alternate preferred embodiment the biophotonic conjugate of the present invention is a bioluminescent conjugate. The subjects of both chemiluminescence and bioluminescence (visible luminescence from living organisms) have been thoroughly studied. Bioluminescent molecules are distinguished from fluorescent molecules in that they do not require the input of radiative energy to emit light. Rather, bioluminescent molecules utilize chemical energy, such as ATP, to produce light. An advantage of bioluminescent moieties, as opposed to fluorescent moieties, is that there is virtually no background in the signal. The only light detected is light that is produced by the exogenous bioluminescent moiety. In contrast, the light used to excite a fluorescent molecule often results in the fluorescence of substances other than the intended target. This is particularly true when the background is as complex as the internal environment of a living animal.

Several types of bioluminescent molecules are known. They include, but are not limited to, the luciferase family and the aequorin family. Members of the luciferase family have been identified in a variety of prokaryotic and eukaryotic organisms. Luciferase and other enzymes involved in the prokaryotic luminescent (lux) systems, as well as the corresponding lux genes, have been isolated from marine bacteria in the Vibrio and Photobacterium genera and from terrestrial bacteria in the Xenorhabdus genus.

An exemplary eukaryotic organism containing a luciferase system (luc) is the North American firefly Photirius pyralls. Firefly luciferase has been extensively studied, and is widely used in ATP assays. Bioluminescent systems such as the firefly luciferase (FFL) system are particularly interesting for biophotonic applications. FFL catalyzes the oxidation of luciferin in the presence of ATP and O₂ to produce oxyluciferin, AMP and green light (λ563 nm). The light produced is very intense, being approximately 1000 times more sensitive than conventional fluorescence techniques. Modified versions of this reaction have been developed utilizing metal ions or coenzyme A to prolong the light emission. cDNAs encoding luciferases from Pyrophorus plagiophthalamus, another species of click beetle, have been cloned and expressed (Wood, et al., Science 244: 700-702 (1989)). This beetle is unusual in that different members of the species emit bioluminescence of different colors. Four classes of clones, having 95-99% homology with each other, were isolated. They emit light at 546 nm (green), 560 nm (yellow-green), 578 nm (yellow) and 593 nm (orange). The last class (593 nm) may be particularly advantageous for use as a light-generating moiety with the present invention, because the emitted light has a wavelength that penetrates tissues more easily than shorter wavelength light.

In a further embodiment, the components of both the capture moiety and biophotonic conjugate are placed on an inert backbone, preferably a polymer. The polymer is a linear polymer or a multibranched polymer such as, but not limited to, star polymers and branched PEGs. The material of the backbone should be inert to the capture moiety and biophotonic conjugate and should have minimal fluorescence and/or luminescence. Furthermore, the backbone should not quench any signal generated by the biophotonic conjugate. Non-limiting examples are PEGs, pluronics, lipids or liposomes. It would also be desirable to have a polymer backbone that is biocompatable and non-toxic to the sample, particularly when the sample comprises living cells or an entire organism such as a mammal.

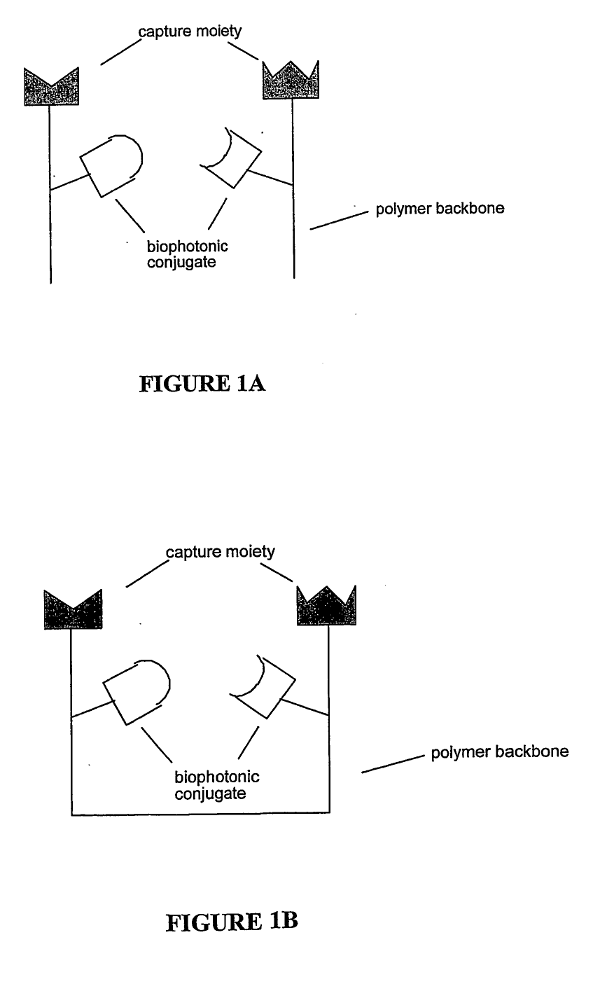

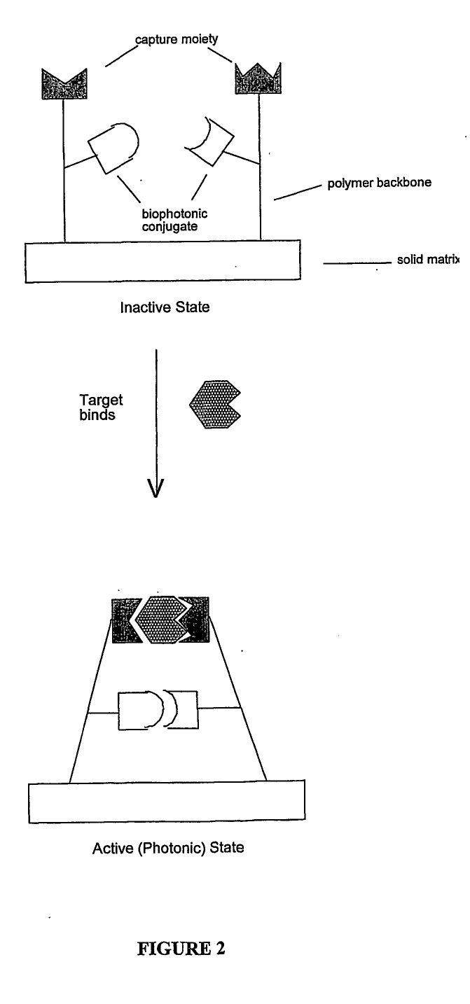

The capture moiety and biophotonic conjugate are attached to the polymer backbone preferably by tethers or side chains on the polymer backbone (FIGS. 1A and 1B). The capture moieties are placed so that they can bind the target molecule and that upon doing so, the polymer backbone assumes a conformation so that the biophotonic conjugates are in the active state. The present invention contemplates arrangements in which, upon binding the target molecule, the biophotonic conjugates are either brought into close proximity to each other or are disassociated and moved apart (FIG. 2). It will be appreciated by those skilled in the art that the arrangement of the biophotonic conjugates will depend on whether or not the components are fluorescent, chemiluminescent or bioluminescent moieties. It will also depend on the photonic properties of the components. It is well known in the art what distances are required for specific components.

In another embodiment, the biophotonic sensor of the present invention has the components of both the capture moiety and biophotonic conjugate on more than one backbone polymer (FIGS. 1A and 2). If the biophotonic sensor has more than one backbone polymer, then at least one of the components of the capture moiety and one of the components of the biophotonic conjugate are attached to a first backbone polymer and at least one of each is attached to a second backbone polymer. In a further aspect of the embodiment, the individual units self assemble into a matrix. Alternately, they can be on a single backbone polymer (FIGS. 1B and 3). In an alternate embodiment, the biophotonic sensor can be in solution or bound to a solid matrix. If the biophotonic sensors are in solution and the active form of the sensor has the components of the biophotonic conjugate in close proximity (FIG. 3), then it is preferable to have the sensor on a single backbone polymer. If the sensor has more than one backbone polymer, then the concentration of the sensor must be high enough or the assay time long enough to account for the diffusion limitation of bringing two separate polymers together. Higher concentrations of the biophotonic sensor may also give higher background signal. This drawback is eliminated if the backbone polymers are attached to a solid matrix or support.

The biophotonic sensors can also be attached to a solid matrix. Non-limiting examples are styrene or glass beads, tubes, multi-well plates or a gel such as, but not limited to a hydrogel. The sensors can be attached to the solid matrices by, but not limited to, hydrophobic interactions, hydrophilic interactions, ionic interactions or covalently bound. Some advantages of using the biophotonic sensors on a solid matrix are the ease of recovery and also the ability to wash away any non-bound material.

Methods for using the biophotonic sensors of the present invention are also provided. The biophotonic sensors can be used in vitro, in vivo, or in situ for detection and/or quantification of biomolecules and compounds of interest. In one embodiment the biophotonic sensors are mixed into a sample to be assayed. The sample may be, but not limited to, a solution, a serum sample or an environmental water sample. Alternatively the sample is cultured cells, an entire organism or a mammal. For example, U.S. Pat. No. 6,217,547 discloses methods for localization of light emitting conjugates in a mammal. However, unlike the biophotonic sensors of the present invention, the conjugates disclosed in the patent do not require binding of a target molecule to produce a signal. If the biophotonic conjugate is fluorescent, the sample is exposed to an energy source of the appropriate wavelength. If the biophotonic sensor does not produce a strong signal until the target molecule is bound, the sample containing is exposed to the energy source of the appropriate wavelength after adding the biophotonic sensor. Alternatively, if the biophotonic sensor produces a measurable fluorescent signal which decreases upon binding of the target molecule, the biosensor is either exposed to the energy source before being added to the sample or after it has been added. The signal generated by the biophotonic compound in solution is then monitored and collected. Non-limiting examples of devices used for collection of the signal may be, but are not limited to fiber optic cables, arrays, waveguides and secondary reporting devices such as signal amplification with ion channels, electronics or other devices.

It is contemplated that the biophotonic sensors of the present invention can be used in methods to identify the presence of specific biomolecules in a mammal. Particularly, the presence of certain viruses in a patient could be detected using the biophotonic sensors. For example, if a patient exhibited symptoms that could be due to either influenza or the common cold, the biophotonic sensor could be used to distinguish between the two causes for the symptoms. A biophotonic sensor having a capture moiety specific for neuramimidase, a viral coat glycoprotein specific to influenza virus, could be introduced into either a blood sample or to the patient themselves. If a signal is detected then the patient has the influenza virus and appropriate treatment can be administered.

The present invention also provides methods for detecting specific cells and or organelles in an organism, particularly a mammal. For example, a biophotonic sensor with a capture moiety specific for receptors or membrane proteins found in a specific tumor cell line may be administered to a patient. The photonic signal from the sensor would allow for the location of tumors in the patient.

The foregoing discussion discloses and describes merely exemplary embodiments of the present invention. One skilled in the art will readily recognize from such discussion, and from the accompanying drawings, that various changes, modifications and variations can be made therein without departing from the spirit and scope of the invention.

All patents and other publications cited herein are expressly incorporated by reference.

1. A biophotonic sensor comprising:
a capture moiety wherein the capture moiety has a high specificity for binding a target molecule; and
a biophotonic conjugate wherein the biophotonic conjugate has a characteristic photonic activity when the target molecule is bound to the capture moiety.

2. The biophotonic sensor of claim 1 wherein the characteristic photonic activity of the biophotonic conjugate is fluorescent, chemiluminescent, or bioluminescent activity.

3. The biophotonic sensor of claim 2 wherein the biophotonic conjugate is bioluminescent.

4. The biophotonic sensor of claim 3 wherein the biophotonic conjugate comprises luciferin and luciferase.

5. The biophotonic sensor of claim 1 wherein the capture moiety is an antibody, an antibody fragment, an enzyme inhibitor, a receptor-binding molecule, a ligand, a toxin or mixtures thereof.

6. The biophotonic sensor of claim 1 wherein the capture moiety and the biophotonic conjugate are attached to a single polymer backbone.

7. The biophotonic sensor of claim 1 wherein one component of the capture moiety and one component of the biophotonic conjugate are attached to a first polymer backbone and a second component of the capture moiety and a second component of the biophotonic conjugate are attached to a second polymer backbone.

8. The biophotonic sensor of claim 1 wherein the biophotonic sensor is attached to a solid matrix.

9. A biophotonic sensor comprising:
a capture moiety wherein the capture moiety has a high specificity for binding a target molecule;
a biophotonic conjugate; and
at least one polymer backbone;
wherein the capture moiety and biophotonic conjugate are attached to the polymer backbone such that when the target molecule is bound to the capture moiety, the biophotonic conjugate has a characteristic photonic activity.

10. The biophotonic sensor of claim 9 wherein the characteristic photonic activity of the biophotonic conjugate is fluorescent, chemiluminescent, or bioluminescent activity.

11. The biophotonic sensor of claim 10 wherein the biophotonic conjugate is bioluminescent.

12. The biophotonic sensor of claim 11 wherein the biophotonic conjugate comprises luciferin and luciferase.

13. The biophotonic sensor of claim 12 wherein the capture moiety is an antibody, an antibody fragment, an enzyme inhibitor, a receptor-binding molecule, a ligand, a toxin or mixtures thereof.

14. The biophotonic sensor of claim 9 wherein the sensor comprises one polymeric backbone, wherein the capture moiety and the biophotonic conjugate are both attached to the one polymeric backbone.

15. The biophotonic sensor of claim 9 wherein the sensor comprises two polymeric backbones wherein one component of the capture moiety and one component of the biophotonic conjugate are attached to a first polymer backbone and a second component of the capture moiety and a second component of the biophotonic conjugate are attached to a second polymer backbone.

16. A method for detecting a target molecule in a sample comprising the steps of:
introducing into the sample the biophotonic sensor of claim 9; and
measuring the photonic activity of the biophotonic sensor.

17. A method for detecting a target molecule in a sample comprising the steps of:
introducing into the sample a biophotonic sensor wherein the biophotonic sensor comprises a capture moiety and a biophotonic conjugate, wherein the biophotonic conjugate has a characteristic photonic activity when the target molecule is bound to the capture moiety; and
measuring the photonic activity of the biophotonic sensor.

18. The method of claim 17 wherein the wherein the characteristic photonic activity of the biophotonic conjugate is fluorescent, chemiluminescent, or bioluminescent activity.

19. The method of claim 18 wherein the biophotonic conjugate is bioluminescent.

20. The method of claim 19 wherein the biophotonic conjugate comprises luciferin and luciferase.

21. The method of claim 17 wherein the capture moiety is an antibody, an antibody fragment, an enzyme inhibitor, a receptor-binding molecule, a ligand, a toxin or mixtures thereof.

22. The method of claim 17 wherein the target molecule is neuromimidase and the sample comprises a biological fluid from a mammal.
